(19) United States
(12) Patent Application Publication
(10) Pub. No.: US 2008/0070952 A1
(43) Pub. Date: Mar. 20, 2008

(54) Inhibition or Treatment of Dyskinesia

(75) Inventors: Mahmoud Irvani, London (GB); Peter Jenner, London (GB)

Correspondence Address: BANNER & WITCOFF, LTD., 1100 13th STREET, N.W., SUITE 1200, WASHINGTON, DC, 20005-4051, US

(73) Assignee: PROXIMAGEN LTD.,, LONDON (GB)

(21) Appl. No.: 11/884,240

(22) PCT Filed: Feb. 20, 2006
(86) PCT No.: PCT/GB06/00583

§ 371 Date: Aug. 13, 2007

(30) Foreign Application Priority Data
Feb. 21, 2005 (GB) 0503540.7
Apr. 25, 2005 (GB) 0508843.0

(51) Int. Cl. A61K 31/44; A61P 25/14

(52) U.S. Cl. 514/343

(57) ABSTRACT

Prevention or treatment of dyskinesia induced by L-dopa or dopamine agonist can be effected by use of a selective inhibitor of neuronal nitric oxide synthase.

The present invention relates to the treatment of dyskinesia in mammals, wherein the dyskinesia is that induced by L-dopa (levodopa; L-3,4-dihydroxyphenylalanine) or dopamine agonist treatment. The present invention also relates to combination therapies for the treatment of Parkinson's disease.

Parkinson's disease is a chronic, progressive disorder of the central nervous system. Parkinson's is thought to be the direct result of the loss of dopamine-producing cells in the substantia nigra section of the brain.

More than 60,000 new cases of Parkinson's are diagnosed in the US each year. The main treatments provide symptomatic relief only. These treatments include the use of dopamine agonists which, whilst effective, may cause nausea, vomiting and psychiatric complications. Dyskinesia is a further possible side effect. Muscarinic antagonists, for example amantidine, are an alternative treatment, but these have been found to be ineffective in the long term.

The most commonly used first line of treatment for Parkinson's is the administration of L-dopa (levodopa). This may be administered in combination with a peripheral decarboxylase inhibitor such as carbidopa which prolongs the effects of L-dopa by slowing the conversion of L-dopa to dopamine in the blood stream. However, long term use of L-dopa causes involuntary movements (dyskinesia) as a significant side effect. In fact, at least 40% of Parkinson's sufferers treated with L-dopa and a peripheral decarboxylase in hibitor develop dyskinesia within 5 years.

The causes of L-dopa induced dyskinesia are currently unknown. It is thought that a priming phenomenon occurs which causes the abnormal movements associated with dyskinesia. The therapeutic strategies for treatment therefore involve both preventing this priming process from occurring, as well as avoiding the expression of dyskinesia in patients where priming has occurred. Possible treatments which have been employed include opioid antagonists (e.g. naloxone) and agonists (e.g. morphine), alpha-2 adrenoceptor antagonists (idozoxan, fipamezole), 5-HT1a receptor agonists, 5-HT2a/c receptor antagonists, cannabinoid receptor agonists (e.g. nabilone), magnesium sulphate, 3,4-methylenedioxymethamphetamine (ecstasy) and levitiracetam.

A common treatment for dyskinesia involves a reduction in, or elimination of, L-dopa (or dopamine agonist) administration. For example a patient may instead be treated with amantidine (a glutamate antagonist). Replacement of L-dopa with amantidine as the anti-Parkinson's therapy relieves the symptoms of dyskinesia, but the benefits are short lasting, since such a treatment regime cannot provide long term relief from Parkinson's. There is thus a need for an effective regime for inhibiting or treating dyskinesia, which can be carried out without affecting anti-Parkinson's treatments.

According top the present invention there is provided the use of a pharmaceutically acceptable selective inhibitor of neuronal nitric oxide synthase (nNOS) in the manufacture of a medicament for use in preventing or treating dyskinesia induced by L-dopa or a dopamine agonist. Also provided is a method of preventing or treating dyskinesia induced by L-dopa or a dopamine agonist in a subject, which method comprises the administration to the said subject of an effective amount of a pharmaceutically acceptable selective inhibitor of neuronal NO synthase.

The invention also comprises an improvement in the treatment Parkinson's Disease in a subject by repeated administration of L-dopa or a dopamine agonist, the improvement comprising administering to the subject an amount of a pharmaceutically acceptable selective inhibitor of neuronal NO synthase effective to reduce dyskinesia in said subject induced by said L-dopa or dopamine agonist.

Three forms of nitric oxide (NO) synthase are known, namely neuronal NO synthase (nNOS), inducible NO synthase (iNOS), and endothelial cell NO synthase (eNOS). All three forms of the enzyme are available commercially as biological reagents. A selective inhibitor of nNOS is one having a lower IC50 against nNOS than against both eNOS and iNOS. Suitable assays for determining the IC50s of a given NOS inhibitor against the three forms of the enzyme is described in Bredt et. al. Proc. Natl. Acad. Sci. USA, Vol 86, pp 9030-9033, November 1989 (for nNOS); Pollock et. al. Proc. Natl. Acad. Sci. USA, Vol 88, pp 10480-10484, December 1991 (for eNOS); and Mahmoud et. alo. J. Biol. Chem. Vol 204, No. 33, Nov. 25, 1989, pp 19654-19658 (for iNOS). The source of the NOS enzyme used in these assays is not critical, and mouse, rat bovine or human enzymes are typically used. Preferably the determination of IC50s for the purpose of establishing nNOS selectivity is made using enzymes from the same species, preferably human.

Preferably the selective nNOS inhibitors for use in the present invention have an IC50 against nNOS at least 10 times and preferably at least 100 times lower than against both eNOS and iNOS.

The selective nNOS inhibitor may be one which does not on its own, without co-administration of L-dopa or a dopamine agonist, represent an effective treatment for the motor deficiency symptoms of dopamine deficiency disease, especially Parkinson's disease. The selective nNOS inhibitor may be one which does not on its own, without co-administration of L-dopa or a dopamine agonist, alter locomotor activity or improve motor deficiency in the animal model described in the Examples herein.

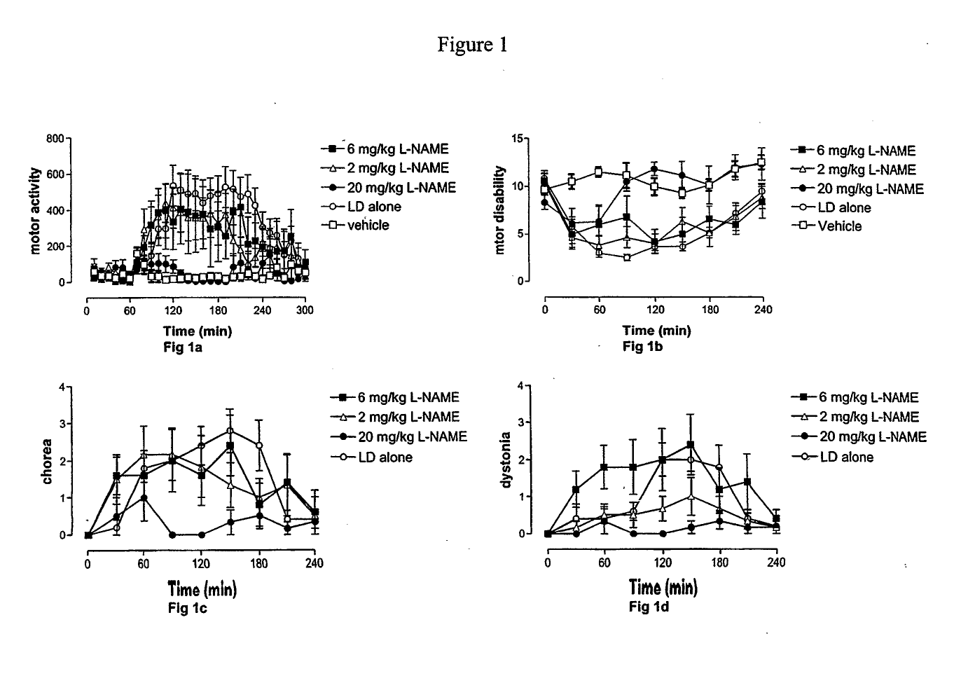

FIG. 1 depicts the results of testing the NOS inhibitor L-Name in the marmoset animal model described below in the Reference Example.

FIG. 1(a) shows the motor activity versus time (min) of Examples 1 to 5.

FIG. 1(b) shows the motor disability versus time (min) of the same five Examples.

FIG. 1(c) depicts chorea versus time (min) for Examples 2 to 5.

FIG. 1(d) depicts dystonia versus time (min) for Examples 2 to 5.

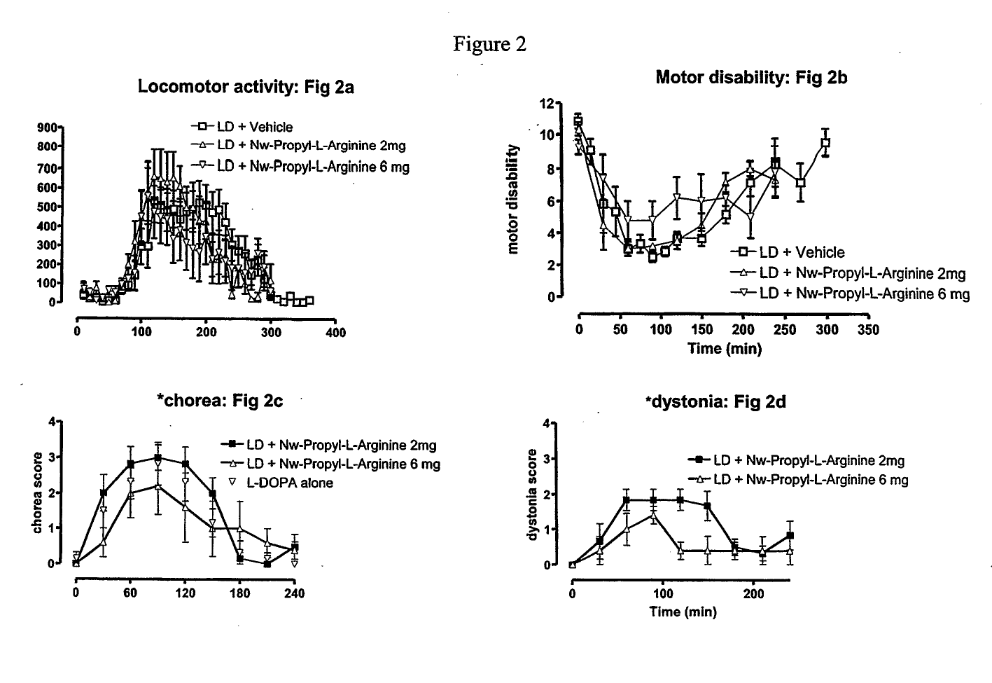

FIG. 2 depicts the results of testing the NOS inhibitor LPA in the marmoset animal model described below in the Reference Example.

FIG. 2(a) shows the motor activity versus time (min) of Examples 2, 6 and 7.

FIG. 2(b) shows the motor disability versus time (min) of the same Examples.

FIG. 2(c) depicts chorea versus time (min) for the same Examples.

FIG. 2(d) depicts dystonia versus time (min) for Examples 6 and 7.

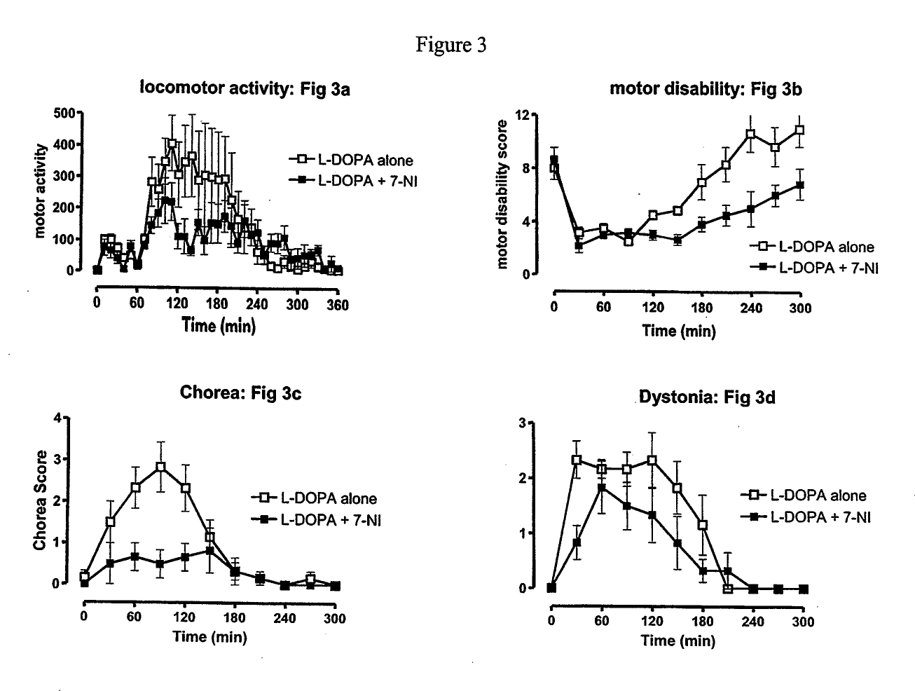

FIG. 3 depicts the results of testing the NOS inhibitor 7-NI in the marmoset animal model described below in the Reference Example.

FIG. 3(a) shows the motor activity versus time (min) of Examples 2 and 8.

For the same Examples, FIG. 3(b) shows the motor disability versus time (min),

FIG. 3(c) depicts chorea versus time (min) and FIG. 3(d) depicts dystonia versus time (min).

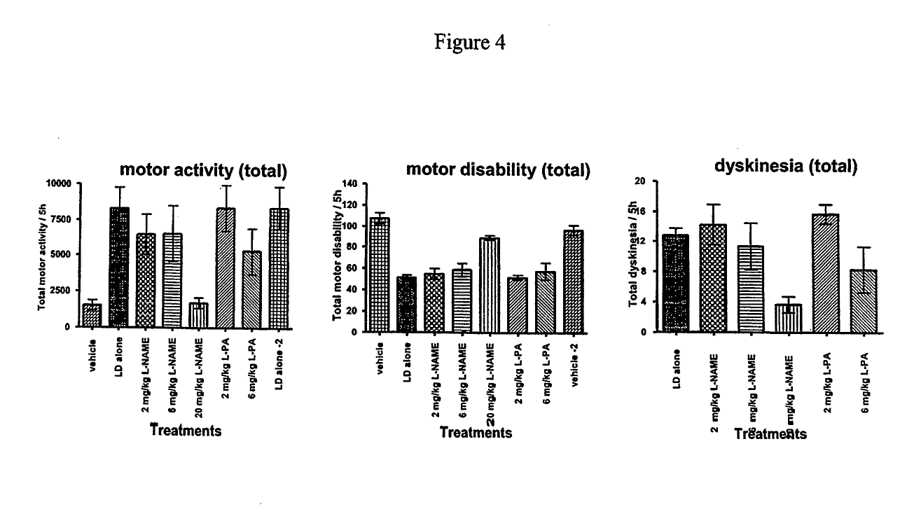

FIG. 4 summarises the results of motor function tests using L-NAME and LPA.

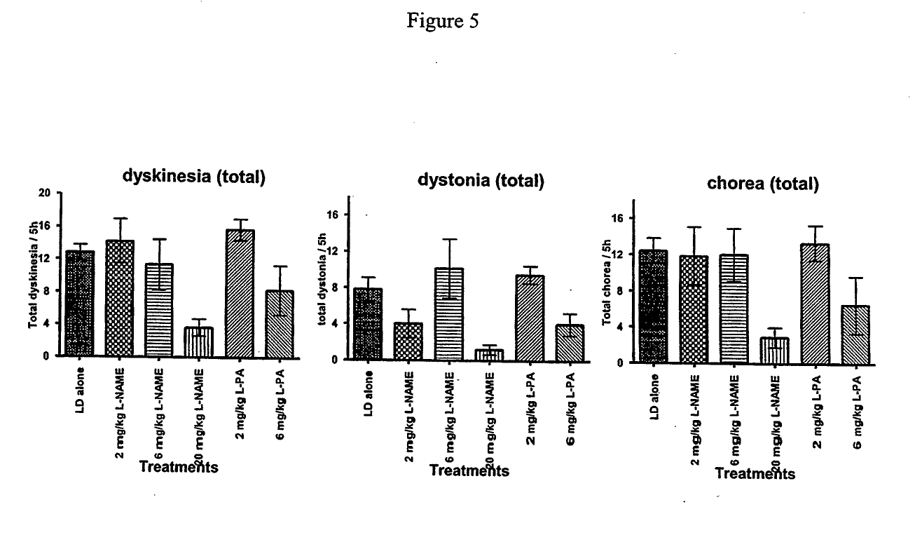

FIG. 5 summarises the results of the total dyskinesia tests using L-NAME and LPA.

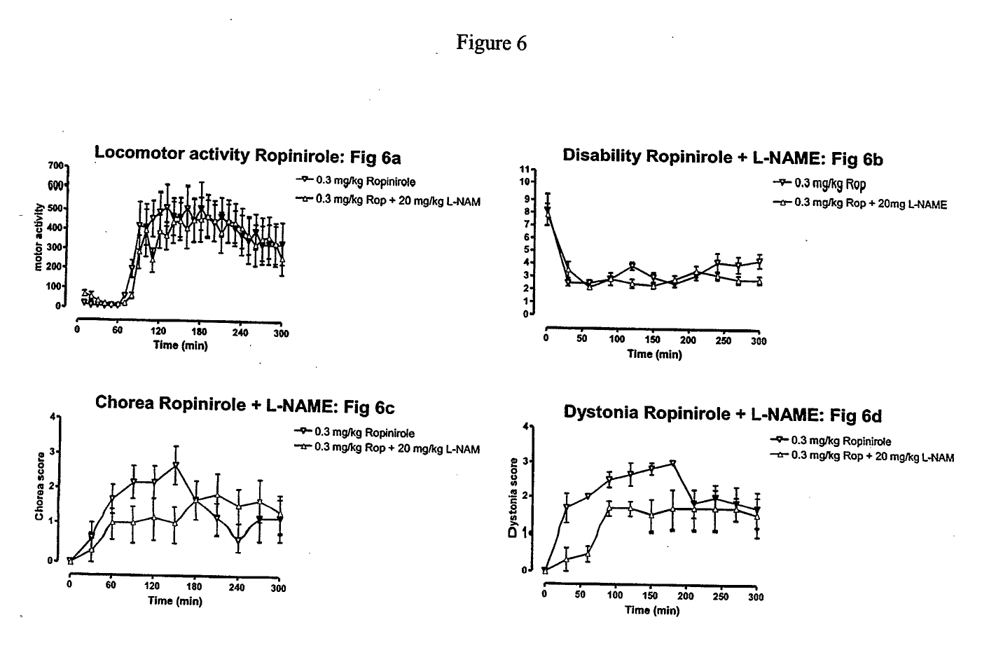

FIG. 6 shows the results of testing the dopamine agonist ropinirole and the NOS inhibitor L-NAME in the marmoset animal model described below in the Reference Example.

FIG. 6(a) shows the locomotor activity in terms of motor activity versus time (min) for Examples 9 and 10.

For the same Examples, FIG. 6(b) shows the results of motor disability tests,

FIG. 6(c) shows the results of chorea tests and

FIG. 6(d) shows the results of dystonia tests.

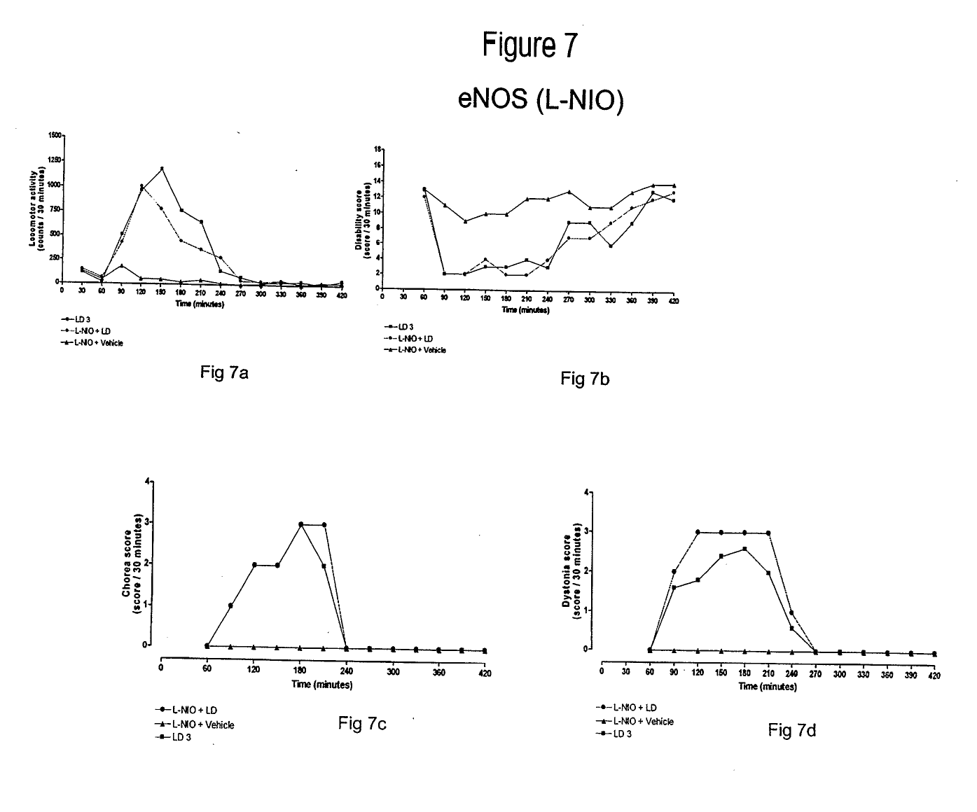

FIG. 7 shows the results of testing the eNOS inhibitor L-NIO in the marmoset animal model described below in the Reference Example.

FIG. 7(a) shows the locomotor activity results for the tests with L-NIO

FIG. 7(b) shows the motor disability results for the tests with L-NIO,

FIG. 7(c) shows the chorea results for the tests with L-NIO, and

FIG. 7(d) shows the dystonia results for the tests with L-NIO,

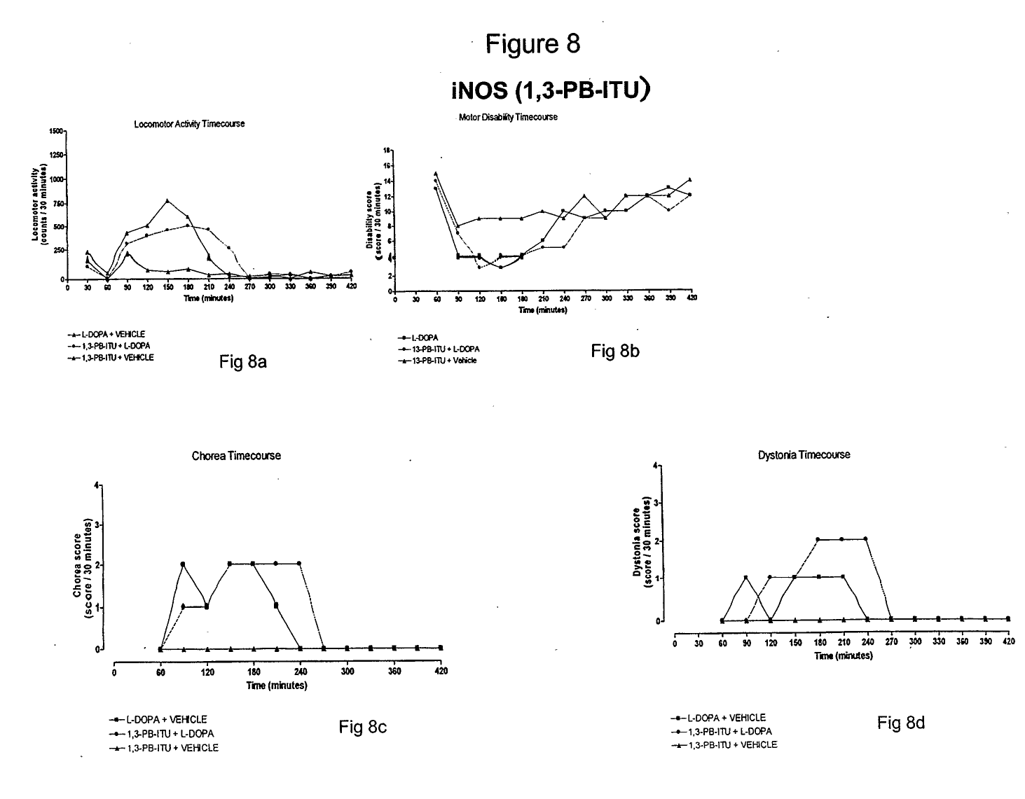

FIG. 8 shows the results of testing the iNOS inhibitor 1,3-PB-ITU in the marmoset animal model described below in the Reference Example.

FIG. 8(a) shows the locomotor activity results for the tests with 1,3-PB-ITU FIG. 8(b) shows the motor disability results for the tests with 1,3-PB-ITU,

FIG. 8(c) shows the chorea results for the tests with 1,3-PB-ITU, and

FIG. 8(d) shows the dystonia results for the tests with 1,3-PB-ITU

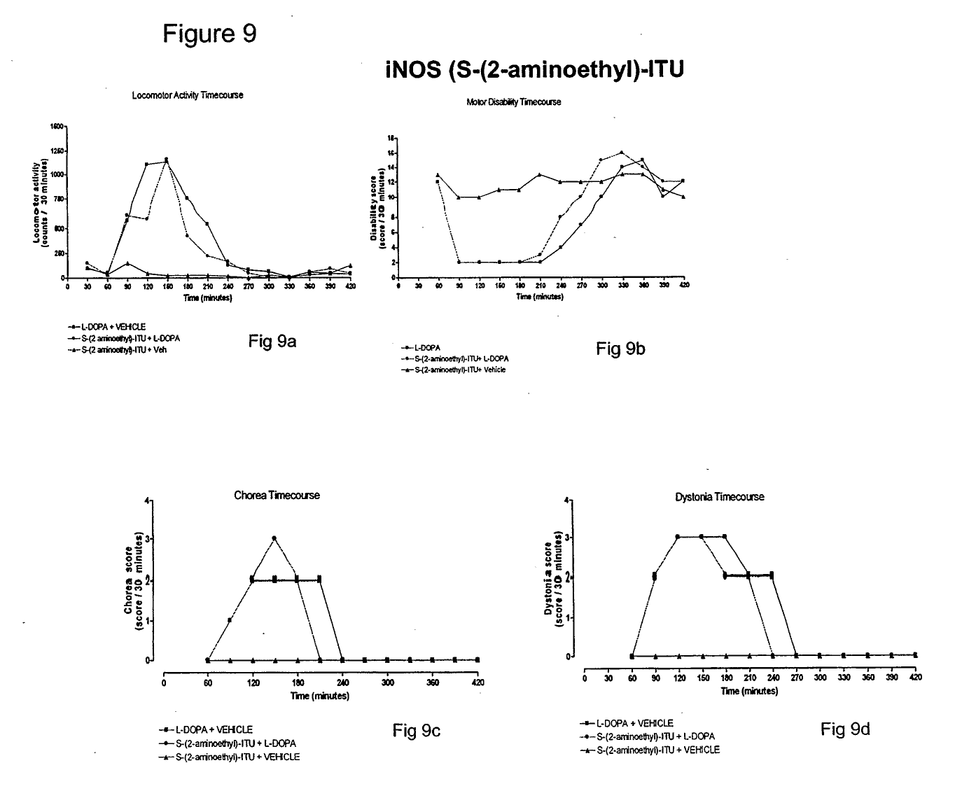

FIG. 9 shows the results of testing the iNOS inhibitor S-(2-aminoethyl)-ITU in the marmoset animal model described below in the Reference Example.

FIG. 9(a) shows the locomotor activity results for the tests with S-(2-aminoethyl)-ITU

FIG. 9(b) shows the motor disability results for the tests with S-(2-aminoethyl)-ITU,

FIG. 9(c) shows the chorea results for the tests with S-(2-aminoethyl)-ITU, and

FIG. 9(d) shows the dystonia results for the tests with S-(2-aminoethyl)-ITU

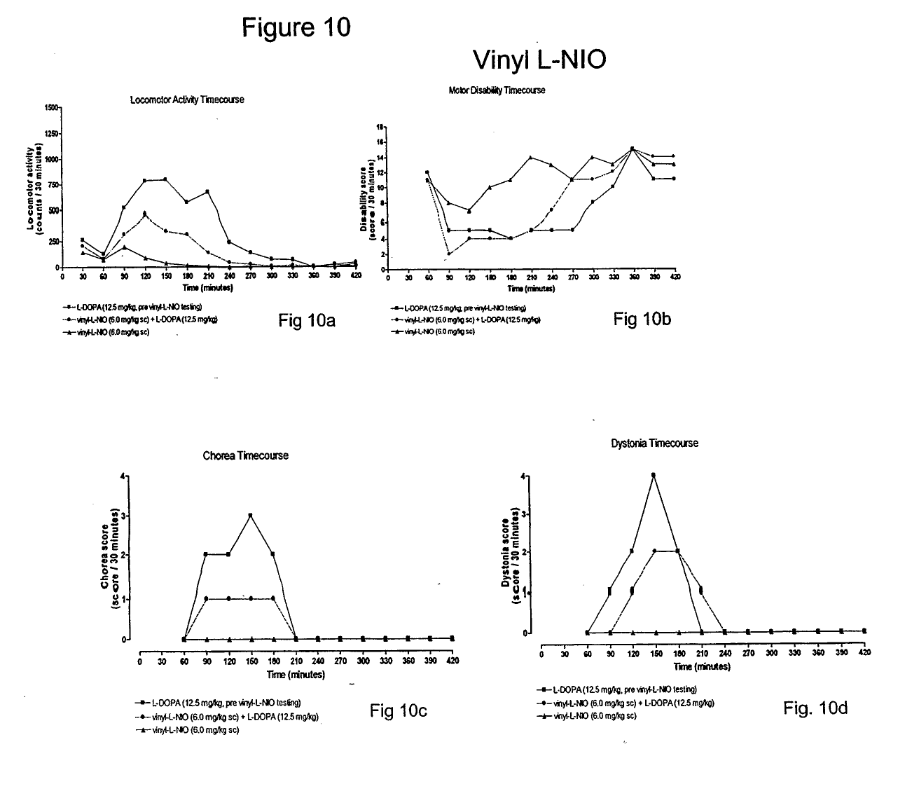

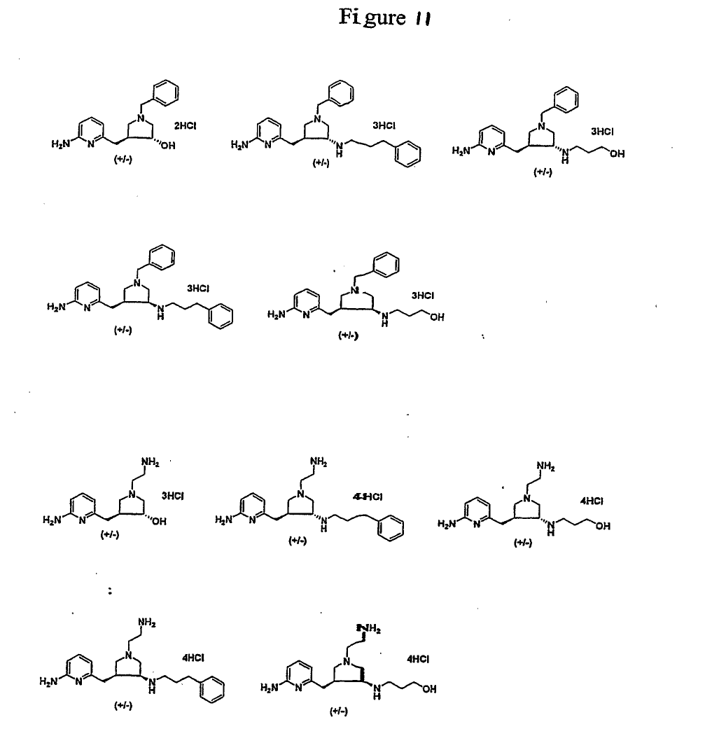

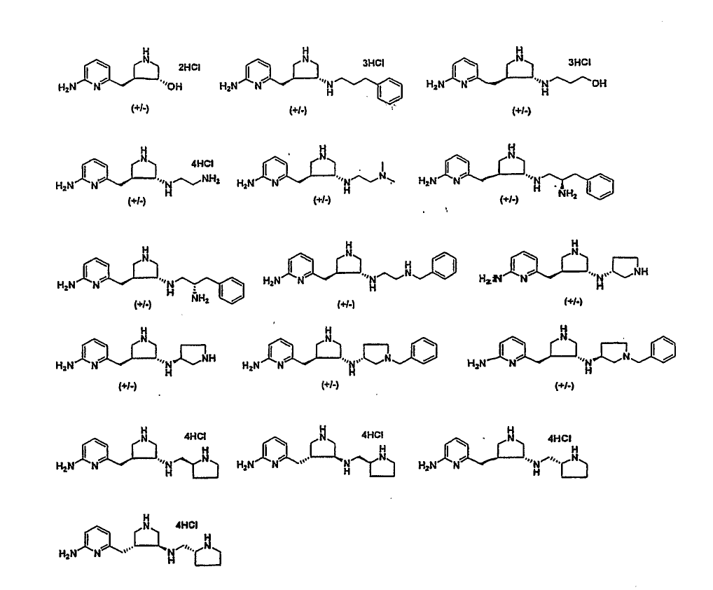

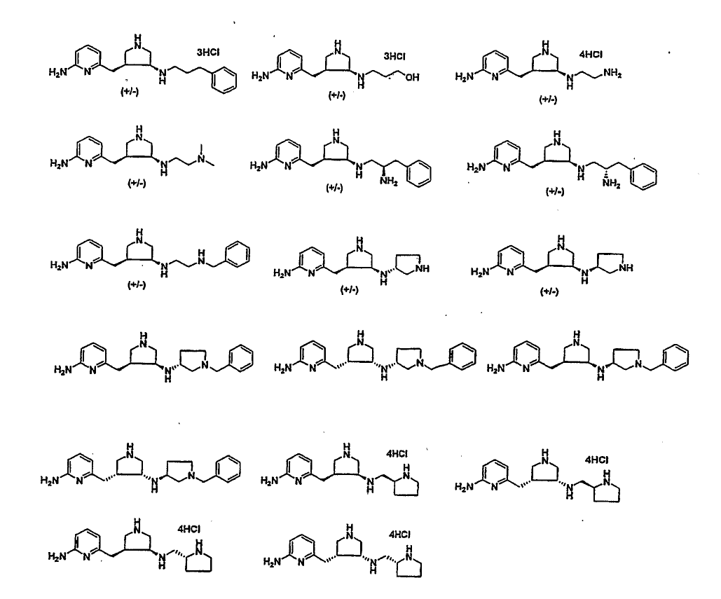

FIG. 10 shows the results of testing the nNOS inhibitor vinyl L-NIO in the marmoset animal model described below in the Reference Example.

FIG. 10(a) shows the locomotor activity results for the tests with vinyl L-NIO

FIG. 10(b) shows the motor disability results for the tests with vinyl L-NIO,

FIG. 10(c) shows the chorea results for the tests with vinyl L-NIO, and

FIG. 10(d) shows the dystonia results for the tests with vinyl L-NIO

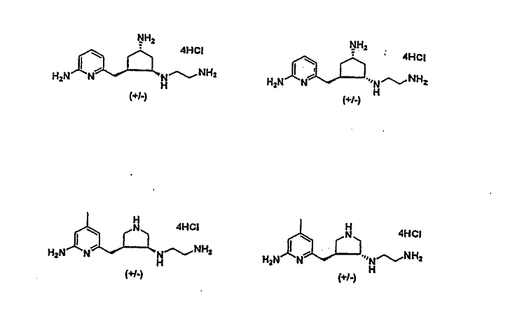

FIG. 11 depicts nNOS inhibitors usable in accordance with the invention.

The present invention is concerned with preventing or treating, in particular preventing, dyskinesia induced by repeated administration of L-dopa or a dopamine agonist. In the context of the present invention, preventing such dyskinesia includes partially preventing such dyskinesia, or reducing the incidence of such dyskinesia. In particular, preventing such dyskinesia includes preventing or reducing the expression of such dyskinesia in mammals primed for dyskinesia.

The invention is particularly useful against dyskinesia induced by L-dopa. In the context of the present invention, dyskinesia induced by L-dopa is dyskinesia which occurs during, or following, administration of L-dopa. Dyskinesia induced by a dopamine agonist is dyskinesia which occurs at the during, or following, administration of a dopamine agonist.

The present invention is useful for the treatment of mammals, in particular humans.

The class of agents whose mode of action is selective inhibition of neuronal NO synthase is a known class, whose numbers are constantly being increasing as research and development of the class continues. Any pharmaceutically acceptable agent having that mode of can be used in the present invention. Competitive, non-competitive, reversible and irreversible inhibitors are suitable.

Selective inhibitors of nNOS are found amongst L-arginine analogues, thiocitrullines, indazole derivatives, aminoguanidine derivatives and thioureas, for example 7-nitroindazole (7-NI).

In one embodiment of the invention, the nNOS in hibitor is a compound of formula 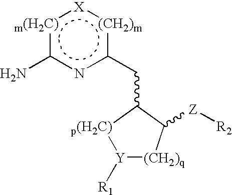
wherein X is selected from CH, N, O and S; m and n are integers independently selected from 0 and 1, and at least one of m and n is 1; Y is selected from CH and N; p and q are integers independently selected from 1 and 2, and at least one of p and q is 1, and p and q are not concurrently 2; Z is selected from NH and O; R₁ is selected from H, alkyl, amino, hydroxyl, and substituted alkyl moieties; and R₂ is selected from alkyl, substituted alkyl, hydroxyalkyl, substituted hydroxyalkyl, aminoalkyl, and substituted aminoalkyl moieties; or a salt thereof.

When R₁ is an alkyl group or a substituted alkyl group, the alkyl group is typically a straight or branched C₁-C₆, preferably C₁-C₄ alkyl group. Typically, when R₁ is substituted alkyl it is aminoalkyl or hydroxyalkyl. Preferably, R₁ is H or aminoalkyl.

Typically, when R₂ is an alkyl group or a substituted alkyl group, the alkyl group is a straight or branched C₁-C₆, preferably C₁-C₄ alkyl group. The alkyl group may also comprise a 5- or 6-membered cycloalkyl group within the alkyl chain.

Typically, when R₂ is substituted alkyl, the substituents are selected from amino (NH₂), hydroxy, C₁-C₄ alkoxy, C₁-C₄ alkylamino, —CONH₂, —CONH(C₁-C₄ alkyl), —COO(C₁-C₄ alkyl), phenyl, pyridyl or phenyl substituted with C₁-C₄ alkyl. Preferably R₂ is alkyl having at least a hydroxy and/or amino substituent. More preferably, R₂ is a primary, secondary or tertiary, linear or cyclic, aminoalkyl group.

In one aspect of this embodiment, X is CH and m and n are 1. In this or other aspects of this embodiment, Y is N, p is 1 or 2 and Z is NH. Alternatively, Y is CH and p and q are 1 or 2, provided at least one of p and q is 1, and preferably both p and q are 1. Alternatively, Y is N and p and q are 1.

In another aspect of this embodiment, X is S and one of m and n is 0. Y, p, q and Z may be as defined above.

In another aspect of this embodiment, the nNOS inhibitor is a compound of formula 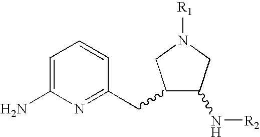
wherein R₁ is selected from H, alkyl, substituted alkyl, aminoalkyl, substituted aminoalkyl, hydroxyalkyl, and substituted hydroxyalkyl moieties; and R₂ is selected from alkyl, substituted alkyl, hydroxalkyl, substituted hydroxyalkyl, aminoalkyl, and substituted aminoalkyl moieties; or a salt thereof. R₁ and R₂ are typically as defined above. Preferably, R₁ is H, alkyl, aminoalkyl or hydroxyalkyl, more preferably H or aminoalkyl. Preferably, R₂ is aminoalkyl.

Examples of nNOS inhibitors in accordance with this embodiment are depicted in FIG. 7. Compounds according to this embodiment can be prepared in accordance with the techniques set out in WO 2005/026111.

In a further embodiment of the invention, the NOS inhibitor is a nitroargenine analogue of the formula 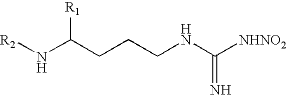
wherein R₂ is H, C₁-C₆ alkyl or prolinyl and R₁ is a group X—R₃ wherein X is —C(O)—, —C(O)—NR₄— or —CH—NR₄—; R₃ is H, a C₁-C₆ alkyl optionally substituted with one or more substituents selected from —NH₂, —CONH₂ and —COOR₅, a pyrrolidinyl or cyclopentyl group optionally substituted with one or more substituents selected from —NH₂—, CONH₂ and —NHCOCF₃, or a group —(CH₂)ₙ-pyridyl or (CH₂)ₘ-phenyl each of which is optionally substituted with one or more substituents selected from —NH₂; —CONH₂ and —(CH₂)ₚ—NH₂; R₄ is H or C₁-C₆ alkyl optionally substituted with one or more substituents selected from —NH₂ and —CONH₂; R₅ is H or C₁-C₄ alkyl; n and m are independently 0, 1 or 2; and p is 1 or 2.

When R₂ is C₁-C₆ alkyl or prolinyl it is unsubstituted or substituted, typically with 1, 2 or 3 substituents. Preferred substituents are —NH₂ and —CONH₂. A prolinyl group is 2-carboxy-pyrrolidinyl. Preferred groups R₂ are H or 4-aminoprolinyl.

X is preferably —C(O)—, —C(O)—NH— or —CHNH—, most preferably —CHNH—.

R₃ is preferably alkyl, pyrrolidinyl, cyclopentyl, —(CH₂)ₙ-pyridyl or —(CH₂)ₘ-phenyl. Preferably, an alkyl, pyrrolidinyl or cyclopentyl group bears 0, 1 or 2 substituents selected from —NH₂ and —CONH₂. When X is —C(O)—, the pyrrolidinyl group is typically linked to X via the N atom. When X is —C(O)—NR₄— or —CH—NR₄—, the pyrrolidinyl group is typically linked to X via a C atom.

When R₃ is (CH₂)ₙ-pyridyl, n is preferably 1 or 2 and the pyridyl group is preferably unsubstituted. When R₃ is (CH₂)ₘ-phenyl, the phenyl group is preferably unsubstituted or substituted with 1 or 2 substituents selected from —NH₂ and —(CH₂)ₚNH₂.

Examples of NOS inhibitors of this embodiment include N-(4S)-{[4-Amino-5-(3-amino)phenylamino]-pentyl}-N′-nitroguanidine, N-(4S)-{[4-Amino-5-(4-amino)phenylamino]-pentyl}-N′-nitroguanidine, N-(4S)-{[4-Amino-5-(2-amino)phenylamino]-pentyl}-N′-nitroguanidine, N-(4S)-[4-Amino-5-(2-aminobenzylamino)pentyl]-N′-nitroguanidine, N-(4S)-[4-Amino-5-(3-aminobenzylamino)pentyl]-N′-nitroguanidine, N-(4S)-[4-Amino-5-(4-aminobenzylamino)pentyl]-N′-nitroguanidine, N-(4S)-{[4-Amino-5-(2-aminophenyl)ethylamino]pentyl}-N′-nitroguanidine, N-(4S)-{[4-Amino-5-(3-aminophenyl)ethylamino]pentyl}-N′-nitroguanidine, N-(4S)-{[4-Amino-5-(4-aminophenyl)ethylamino]pentyl}-N′-nitroguanidine, N-(4S)-{4-Amino-5-[(2-aminomethyl)phenylamino]pentyl}-N′-nitroguanidine, N-(4S)-{4-Amino-5-[(3-aminomethyl)phenylamino]pentyl}-N′-nitroguanidine, N-(4S)-{4-Amino-5-[(4-aminomethyl)phenylamino]pentyl}-N′-nitroguanidine, N-(4S)-{4-Amino-5-[2-(2-aminoethyl)phenylamino]-pentyl}-N′-nitroguanidine, N-(4S)-{4-Amino-5-[3-(2-aminoethyl)phenylamino]pentyl}-N′-nitroguanidine, N-(4S)-{4-Amino-5-[4-(2-aminoethyl)phenylamino]pentyl}-N′-nitroguanidine, N-(4S)-{4-Amino-5-[(pyridin-2-yl)methyl]aminopentyl}-N′-nitroguanidine, N-(4S)-{4-Amino-5-[(pyridin-3-yl)methyl]amino]pentyl-N′-nitroguanidine, N-(4S)-{4-Amino-5-[(pyridin-4-yl)methyl]aminopentyl}-N′-nitroguanidine, N-(4S)-{4-Amino-5-[(2-pyridin-2-yl)ethyl]aminopentyl}-N′-nitroguanidine, N-(4S)-{4-Amino-5-[(2-pyridin-3-yl)ethyl]aminopentyl}-N′-nitroguanidine, N-(4S)-{4-Amino-5-[(2-pyridin-4-yl)ethylamino]pentyl}-N′-nitroguanidine, L-nitroArg-NH—(CH₂)₂—NH₂, L-nitroArg-NH—(CH₂)₃—NH₂, L-nitroArg-NH—(CH₂)₄—NH₂, L-nitroArg-NH—CH(CONH₂)—(CH₂)₂—NH₂, L-nitroArg-NH—CH(CONH₂)—(CH₂)₃—NH₂ and L-nitroArg-NH—CH(CONH₂)—(CH₂)₄—NH₂.

The NOS inhibitors of this embodiment can be prepared in accordance with the methods described in U.S. Pat. No. 6,803,486.

In a further embodiment of the invention, the NOS inhibitor is a dipeptide containing at least one, preferably one, N^{w}-nitroArg or other unnatural amino acid residue. The unnatural amino acid residue can be located at either the N- or C-terminus of the dipeptide and can be in either the D- or L-configuration. In a preferred aspect of this embodiment, the N-terminal residue is N^{w}-L-nitroArg. In another preferred aspect of this embodiment, the C-terminal residue is N^{w}-D-nitroArg. In either case, the second amino acid preferably has an —NH₂ group on its side chain. Where there is only one unnatural amino acid residue, the other residue can be any natural amino acid residue. The other residue can also be in either the D- or L-configuration. The dipeptide can be in the form of an ester, an amide, or other peptidomimetic group.

Examples of the unnatural amino acid residues include nitroLys, ornithine (Orn), 2-4-diaminobutanoic acid (Dbu), 2,3-diaminopropanoic acid (Dpr) and substituted arginines. The substituent group on Arg is preferably a C₁-C₆ alkyl or C₂-C₆ alkenyl group. The substituent group can be linear, branched or cyclic. A preferred substituent group for Arg is C₁-C₆ alkyl.

In one preferred aspect of this embodiment, the dipeptide contains an N^{w}-nitroArg residue and Phe. Dipeptides having L-nitroArg at the N-terminus and methyl ester dipeptides containing a D amino acid are preferred as they are selective inhibitors of nNOS. Especially preferred dipeptides that contain nitroArg and Phe are D-Phe-L-nitroArg, L-nitroArg-L-Phe, L-nitroArg-L-Phe-OMe, L-nitroArg-L-Phe-OMe, D-nitroArg-D-Phe-OMe, D-Phe-L-nitroArg-OMe, L-Phe-D-nitroArg-OMe, or D-Phe-D-nitroArg-OMe. Further Examples of dipeptides that contain nitroArg and Phe include L-Phe-L-nitroArg, L-Phe-L-nitroArg-OMe, L-nitroArg-L-Phe-OBn, L-Phe-L-nitroArg-OBn, L-nitroArg-D-Phe-OMe, D-nitroArg-L-Phe-OMe and D-Phe-D-nitroArg.

In another aspect of this embodiment, the dipeptide is a dipeptide amide that contains one N^{w}-nitroArg and a second residue other than N^{w}-nitroArg or Phe. Especially preferred such peptides are N^{w}-L-nitroArg-L-Lys, N-L-nitroArg-N^{w}-L-nitroArg, L-Lys-N^{w}-D-nitroArg, D-Lys-N^{w}-D-nitroArg, L-His-N-D-nitroArg, N^{w}-L-nitroArg-D-Glu, N^{w}-D-nitroArg-L-Ser, and N^{w}-L-nitroArg-D-Asn, N^{w}-L-nitroArg-L-Dpr, N^{w}-nitroArg-L-Orn, N^{w}-L-nitroArg-D-Orn, L-Dbu-N^{w}-D-nitroArg, L-Orn-N^{w}-D-nitroArg and D-Orn-N^{w}-D-nitroArg. Preferably the N^{w}-nitroarginine is L-N^{w}-nitroarginine.

In another aspect of this embodiment, the dipeptide contains N-nitroArg and 2,4-diaminobutyric acid (Dbu). Examples of these dipeptides include L-nitroArg-L-Dbu, L-nitroArg-D-Dbu, D-nitroArg-D-Dbu, D-Dbu-L-nitroArg, D-Dbu-D-nitroArg and D-nitroArg-L-Dbu.

In a further aspect of this embodiment, the dipeptide contains one N-nitroArg residue and an amino-substituted proline residue coupled to the N or C, preferably the N, terminus of the N-nitroArg residue. Preferred such dipeptides include L-nitroArg-4-amino-Pro, in particular 4N-(L-nitroArg)-L-trans-4-nitroPro and 4N(L-nitroArg)-D-trans-4-nitroPro.

The dipeptides described in this embodiment can be prepared by methods well known to the skilled person, for example as described in U.S. Pat. No. 6,274,557 or U.S. Pat. No. 6,803,486.

In a further embodiment of the invention, the NOS inhibitor is an amidine. Examples of suitable amidines include N-(1-iminoethyl)-L-ornithine, N-(imino-3-butenyl)-L-ornithine, N-(1-iminoethyl)-L-lysine, 2-amino-6-(1-imino-2-fluoroethylamino)-4,4-dioxo-4-thiohexanoic acid, N-(3-(aminomethyl)phenyl)acetamidine, N-(3-(aminoethyl)phenyl)acetamidine, N-(3-(aminomethyl)benzyl)acetamidine and N,N′-(1,3-benzyl)bisamidine. Of these, N-(3-(aminomethyl)phenyl)acetamidine and N-(imino-3-butenyl)-L-ornithine are selective for nNOS and are accordingly preferred.

In a further aspect of this embodiment, the NOS inhibitor is a compound of formula 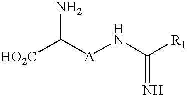
wherein R₁ is NH₂, NHCH₃ or CH₃, preferably NH₂, and A is —CH₂CH═CH—, phenyl or phenyl-CH₂— wherein the left-hand end of the group A as written is bound to the NH in the above formula.

In a further aspect of this embodiment, the NOS inhibitor is a compound of formula 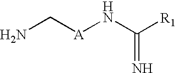
wherein R₁ is furanyl or thiophenyl and A is phenyl.

The amidines of this embodiment can be produced in accordance with techniques known in the art and some are natural products. Further techniques for producing the amidines of this embodiment are discussed by Lee et al (Bioorg & Med. Chem, 1999, 7, 1097-1104).

In a further embodiment of the invention, the NOS inhibitor is a pyrazole derivative of formula 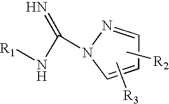
wherein R₁ is H, C₁-C₆ alkyl, C₃-C₆ cycloalkyl or phenyl, R₁ being unsubstituted or substituted with one or two substituents selected from NH₂ and, in the case that R₁ is phenyl, (CH₂)ₙNH₂ wherein n is 1 or 2;
R₂ and R₃ are independently hydrogen or methyl, preferably hydrogen.

Preferred inhibitors in this embodiment of the invention are those wherein R₂ and R₃ are H and R₁ is H, C₁-C₄alkyl optionally substituted with NH₂ or phenyl substituted with one NH₂ or (CH₂)ₚNH₂ substituent. Particularly preferred inhibitors are those wherein R₂ and R₃ are H and R₁ is H, propyl, —(CH₂)₄—NH₂ or 2-aminomethylphenyl.

In a further embodiment of the invention, the NOS inhibitor is an imizadole-containing amino acid of the formula 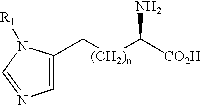
wherein R₁ is H or phenyl and n is 0, 1, 2, 3 or 4.

In another embodiment the nNOS inhibitor is one of those referred to in: WO 97/36871; WO 2004/073712; U.S. provisional patent application 60/057,739; WO98/34919, and WO 98/24766. Other examples of NOS inhibitors of this embodiment are described in WO 03/030993, U.S. Pat. No. 6,235,747, U.S. Pat. No. 6,465,491 and U.S. Pat. No. 6,803,470. The foregoing patent applications and patents are each incorporated by reference herein in their entireties.

Preferred nNOS inhibitors of this embodiment include those formula VIII 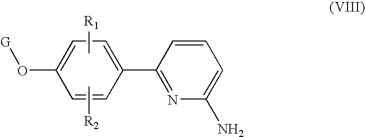
wherein R¹ and R² are selected, independently, from hydrogen, halo, hydroxy, (C₁-C₆) alkoxy, (C₁-C₇) alkyl, (C₂-C₆) alkenyl, and (C₂-C₁₀) alkoxyalkyl; and G is selected from hydrogen, (C₁-C₆)alkyl, (C₁-C₆)alkoxy-(C₁-C₃)alkyl, aminocarbonyl-(C₁-C₃)alkyl-, (C₁-C₃)alkylaminocarbonyl-(C₁-C₃)alkyl-, di-[(C₁-C₃)alkyl]aminocarbonyl-(C₁-C₃)alkyl-, and N(R³) (R⁴)(C₀-C₄)alkyl-, wherein R³ and R⁴ are selected, independently, from hydrogen, (C₁-C₇)alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₇)alkyl and said tetrahydronaphthalene and the aryl moiety of said aralkyl may optionally be substituted with from one to three substituents, preferably from zero to two substituents, that are selected, independently, from halo, nitro, hydroxy, cyano, amino, (C₁-C₄) alkoxy, and (C₁-C₄) alkylamino;
or R³ and R⁴ form, together with the nitrogen to which they are attached, a piperazine, piperidine, azetidine or pyrrolidine ring or a saturated or unsaturated azabicyclic ring system containing from 6 to 14 ring members, from 1 to 3 of which are nitrogen, from zero to two of which are oxygen, and the rest of which are carbon;
and wherein said piperazine, piperidine, azetidine and pyrrolidine rings and said azabicyclic ring systems may optionally be substituted with one or more substituents, preferably with from zero to two substituents, that are selected, independently, from (C₁-C₆)alkyl, amino, (C₁-C₆)alkylamino, [di-(C₁-C₆)alkyl]amino, phenyl substituted 5 to 6 membered heterocyclic rings containing from 1 to 4 ring nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;
and wherein said piperazine, piperidine, azetidine and pyrrolidine rings and said azabicyclic ring systems may be attached to —(C₀-C₄)alkyl-O— at a nitrogen atom of the NR³R⁴ ring or at any other atom of such ring having an available bonding site; or G is a group of the formula A 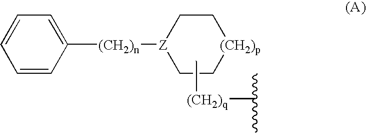
wherein Z is nitrogen or CH, n is zero or one, q is zero, one, two or three and p is zero, one or two;
and wherein the 2-amino piperidine ring depicted in structure I above may optionally be replaced with 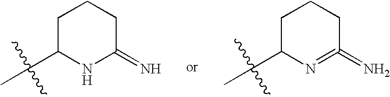
and the pharmaceutically acceptable salts of such compounds.

Other preferred nNOS inhibitors useful in this embodiment are compounds of formula (VI) 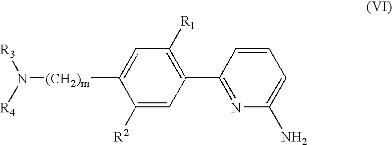
wherein R¹ is selected from methyl, ethyl, propyl, butyl, isopropyl, 2-methylpropyl, t-butyl, methoxy, ethoxy, and propoxy;
R² is selected from hydrogen, methyl, ethyl, propyl, butyl, isopropyl, 1-methylpropyl, 2-methylpropyl, t-butyl, methoxy, ethoxy, and propoxy;
m is one, two or three;
R³ and R⁴ are selected, independently, from R⁷; phenyl; 5 or 6 membered heteroaryl containing from 1 to 4 heteroatoms independently selected from O, N, and S; and straight chain or branched (C₁-C⁶)alkyl substituted with from 1 to 3 substituents selected independently from R⁶, —CF₃, halo, (i.e. bromine, chlorine, iodine, and fluorine), —NR⁷R⁸, (C₃-C₆)cycloalkyl, 3 to 9 membered heterocycloalkyl containing 1 or 2 heteroatoms independently selected from O, N, and S, phenyl, and or 6 membered heteroaryl containing from 1 to 4 heteroatoms independently selected from O, N, and S;
wherein said phenyl, heteroaryl, cycloalkyl, and heterocycloalkyl groups of R³ and R₄ are optionally independently substituted with from 1 to 3 substituents independently selected from R⁶ and straight chain or branched C₁-C₆ alkyl optionally comprising 1 or 2 double or triple bonds;
or R³ and R⁴ are connected, with the nitrogen atom to which they are attached, to form a 3 to 9 membered heterocyclic ring, which heterocyclic optionally comprises from one to three heteroatoms in addition to said nitrogen atom, which optional heteroatoms are selected independently from O, S, and N;
wherein said heterocyclic ring formed by R³ and R⁴ optionally is fused to form a fused ring system with one or two aromatic rings selected independently from benzene rings and heteroaromatic rings, which aromatic rings share two carbon atoms with said heterocyclic ring; or which heterocyclic ring formed by R³ and R⁴ is optionally fused to form a fused or spiro ring system to a 3 to 8 membered carbocyclic ring which shares one or two carbon atoms with said heterocyclic ring; wherein fused or spiro ring systems contain up to 15 ring members;
and wherein said heterocyclic ring, said optional aromatic rings, and said optional carbocyclic ring, are each optionally and independently substituted with from 1 to 3 substituents independently selected from R⁶, —O—(C₁-C₆ alkyl)-R⁶, —S—(C₁-C₆ alkyl)-R⁶, straight chain or branched (C₁-C₆)alkyl optionally substituted with R⁶, —C(═O)O—((C₁-C₆)alkyl), 3 to 6 membered cycloalkyl, phenyl, benzyl, and 5 or 6 membered heteroaryl; wherein said cycloalkyl, phenyl, benzyl, and heteroaryl are independently optionally substituted with from 1 to 3 substituents independently selected from R⁵;
R⁵ is selected from R⁶, straight chain or branched (C₁-C₆ alkyl), —(C₁-C₆ alkyl)-R⁶, and 5 or 6 membered heteroaryl optionally substituted with 1 or 2 substituents independently selected from R⁶, —NR⁷R⁸, straight chain or branched (C₁-C₆)alkyl, and (C₁-C₆)alkyl-R⁶;
R⁶ is selected from —O—R⁷ and —S—R⁷;
R⁷ is selected from H and straight chain or branched (C₁-C₆)alkyl (e.g. methyl, ethyl, propyl, butyl, isopropyl, 1-methylpropyl, 2-methylpropyl, t-butyl, pentyl, 3-methylbutyl, 1,2-dimethylpropyl, or 1,1-dimethylbutyl) optionally comprising 1 or 2 double or triple bonds; and R⁸ is selected from H and straight chain or branched (C₁-C₆) alkyl; and pharmaceutically acceptable salts thereof.

Other preferred nNOS inhibitors useful in this embodiment are the following compounds and their pharmaceutically acceptable salts:
(a) 6-[4-(N-methyl-3-azetidinoxy)-5-ethyl-2-methoxy-phenyl]-pyridin-2-ylamine, which h as the following structure 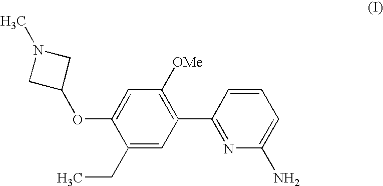
(b) 6-[4-(N,N-dimethylaminomethyl)-5-ethyl-2-methoxy-phenyl]-pyridin-2-ylamine, which has the following structure 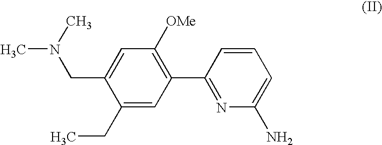
(c) 6-[4-(N-methylaminomethyl)-5-ethyl-2-methoxy-phenyl]-pyridin-2-ylamine, which has the following structure 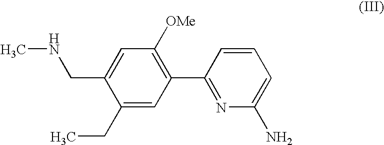
(d) 6-[4-(3-azetidin oxy)-5-ethyl-2-methoxy-phenyl]-pyridin-2-ylamine, which has the following structure 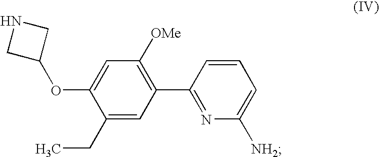
(e) 6-[4-(2-dimethylamino-ethoxy)-5-ethyl-2-methoxy-phenyl]-pyridin-2-ylamine, which has the following structure

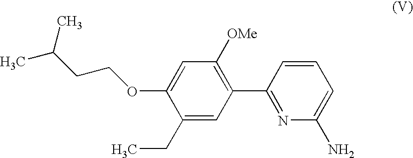

Other examples of NOS inhibitors that can be used in this embodiment are compounds of the formula 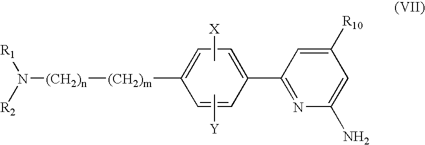
wherein R¹ and R² are selected, independently, from (C₁-C₆)alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₆)alkyl and said tetrahydronaphthalene and the aryl moiety of said aralkyl may optionally be substituted with from one to three substituents, preferably from zero to two substituents, that are selected, independently, from halo (e.g., chloro, fluoro, bromo, iodo), nitro, hydroxy, cyano, amino, (C₁-C₄)alkoxy, and (C₁-C₄)alkylamino;
or R¹ and R²m form, together with the nitrogen to which they are attached, a piperazine, piperidine or pyrrolidine ring or an azabicyclic ring containing from 6 to 14 ring members, from 1 to 3 of which are nitrogen and the rest of which are carbon, wherein examples of said azabicyclic rings are the following 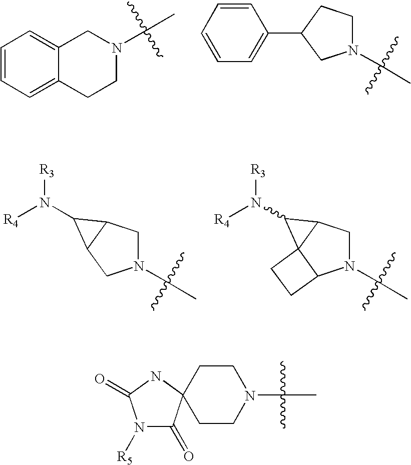
wherein R³ and R⁴ are selected from hydrogen, (C₁-C₆)alkyl, phenyl, naphthyl, (C₁-C₆)alkyl-C(═O)—, HC(═O)—, (C₁-C₆)alkoxy-(C═O)—, phenyl-C(═O)—, naphthyl-C(═O)—, and —(R⁷)₂NC(═O) wherein each R⁷ is selected, independently, from hydrogen and (C₁-C₆)alkyl; R⁵ is selected from hydrogen, (C₁-C₆)alkyl, phenyl, napthyl, phenyl-(C₁-C₆)alkyl- and naphthyl(C₁-C₆)alkyl-;
and wherein said piperazine, piperidine and pyrorrolidine rings may optionally be substituted with one or more substituents, preferably with from zero to two substituents, selected independently, from (C₁-C₆)alkylamino, [di(C₁-C₆)alkyl]amino, phenyl substituted 5 to 6 membered heterocyclic rings containing from 1 to 4 ring nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃; n is 0, 1 or 2; and each carbon of said (CH₂)ₙ can optionally be substituted with a substitutent R⁸;
m is 0, 1, or 2; and each carbon of said (CH₂)ₘ can optionally be substituted with a substitutent R⁹; (C₁-C₄)alkyl, aryl-(C₁-C₄)alkyl wherein said aryl is selected from phenyl and naphthyl; allyl and phenallyl;
X and Y are selected, independently, from methyl, methoxy, hydroxy and hydrogen; and R¹⁰ is H(C₁-C₆)alkyl;
with the proviso that R⁸ is absent when n is zero and R⁹ is absent when m is zero.

Compounds of formula VII are disclosed, and their synthesis described, in U.S. Pat. No. 6,235,747, and U.S. Pat. No. 6,465,491, both mentioned above and incorporated herein by reference.

Other nNOS inhibitors that are useful in this embodiment are compounds of formula 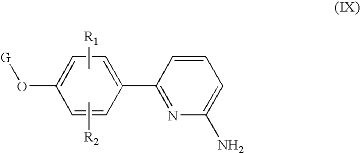
wherein R¹ and R² are selected, independently, from hydrogen, halo, hydroxy, (C₁-C₆)alkoxy, (C₁-C₇)alkyl, (C₂-C₆)alkenyl, and (C₂-C₁₀)alkoxyalkyl; and G is selected from hydrogen, (C₁-C₆)alkyl, (C₁-C₆)alkoxy-(C₁-C₃)alkyl, aminocarbonyl-(C₁-C₃)alkyl-, (C₁-C₃)alkylaminocarbonyl-(C₁-C₃)alkyl-, di-[(C₁-C₃)alkyl]aminocarbonyl-(C₁-C₃)alkyl-, and N(R³)(R⁴)(C₀-C₄)alkyl-, wherein R³ and R⁴ are selected, independently, from hydrogen, (C₁-C₇)alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₇)alkyl and said tetrahydronaphthalene and the aryl moiety of said aralkyl may optionally be substituted with from one to three substituents, preferably from zero to two substituents, that are selected, independently, from halo, nitro, hydroxy, cyano, amino, (C₁-C₄)alkoxy, and (C₁-C₄)alkylamino;
or R³ and R⁴ form, together with the nitrogen to which they are attached, a piperazine, piperidine, azetidine or pyrrolidine ring or a saturated or unsaturated azabicyclic ring system containing from 6 to 14 ring members, from 1 to 3 of which are nitrogen, from zero to two of which are oxygen, and the rest of which are carbon; and wherein said piperazine, piperidine, azetidine and pyrrolidine rings and said azabicyclic ring systems may optionally be substituted with one or more substituents, preferably with from zero to two substituents, that are selected, independently, from (C_{I}-C₆)alkyl, amino, (C₁-C₆)alkylamino, [di-(C₁-C₆)alkyl]amino, phenyl substituted 5 to 6 membered heterocyclic rings containing from 1 to 4 ring nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;
and wherein said piperazine, piperazine, piperidine, azetidine and pyrrolidine rings and said azabicyclic ring systems may be attached to —(C₀-C₄)alkyl-O— at a nitrogen atom of the NR³R⁴ ring or at any other atom of such ring having an available bonding site;
or a group of formula A 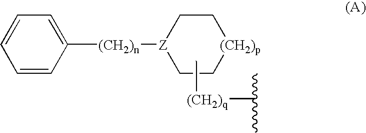
wherein Z is nitrogen or CH, n is zero or one, q is zero, one, two or three and p is zero, one or two;
and wherein the 2-amino piperidine ring depicted in structure I above may optionally be replaced with 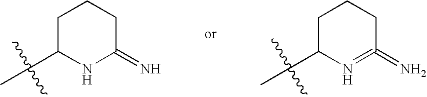
and the pharmaceutical acceptable salts of such compounds.

The compounds of formula IX are disclosed and their synthesis described in U.S. Ser. No. 09/127,158, mentioned and incorporated herein by reference above.

Except as otherwise indicated, the above NOS inhibitors are commercially available, or may be made by analogy with known methods.

The inhibitor may be a pharmaceutically acceptable salt of one the above compounds. Suitable salts include salts with pharmaceutically acceptable acids, both inorganic acids such as hydrochloric, sulphuric, phosphoric, diphosphoric, hydrobromic or nitric acid and organic acids such as citric, fumaric, maleic, malic, ascorbic, succininc, tartaric, benzoic, acetic, methanesulphonic, ethanesulphonic, benzenesulphonic or p-toluenesulphonic acid. Salts may also be formed with pharmaceutically acceptable bases such as alkali metal (eg sodium or potassium) and alkali earth metal (eg calcium or magnesium) hydroxides and organic bases such as alkyl amines, aralkyl amines or heterocyclic amines.

The selective inhibitors of nNO synthase may be administered in a variety of dosage forms. Thus, they can be administered orally, for example as tablets, troches, lozenges, aqueous or oily suspensions, dispersible powders or granules. The inhibitors may also be administered parenterally, either subcutaneously, intravenously, intramuscularly, intrasternally, transdermally or by infusion techniques. The inhibitors may be administered intranasally or by inhalation. The inhibitors may also be administered as suppositories.

A selective nNO synthase inhibitor is typically formulated for administration in the present invention with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusions may contain as carrier, for example, sterile water or preferably they may be in the form of sterile, aqueous, isotonic saline solutions.

A therapeutically effective amount of a selective nNO synthase inhibitor is administered to a patient. A typical daily dose is from about 0.1 to 50 mg per kg of body weight, according to the activity of the specific inhibitor, the age, weight and conditions of the subject to be treated, the type and severity of the dyskinesia, any simultaneous anti-Parkinson's treatments and the frequency and route of administration. Preferably, daily dosage levels are from 1 mg to 50 mg, for example from 2 to 30 mg, e.g. 5 to 20 mg or 8 to 20 mg. The selective nNOS inhibitor may be administered at a dose which, when the inhibitor is administered alone without co-administration of L-dopa or a dopamine agonist, does not on its own represent an effective treatment for the motor deficiency symptoms of dopamine deficiency disease, especially Parkinson's disease. The selective nNOS inhibitor may be may be administered at a dose which, when the inhibitor is administered alone without co-administration of L-dopa or a dopamine agonist, does not alter locomotor activity or improve motor deficiency in the animal model described in the Examples herein.

In one embodiment of the invention, (a) L-dopa or (b) a dopamine agonist or (c) both L-dopa and a dopamine agonist are administered in combination with a selective inhibitor of nNO synthase. In this embodiment, treatment of Parkinson's disease is effected by administration of L-dopa and/or dopamine agonist, whilst the potential side effect of dyskinesia is inhibited by the administration of the nNO synthase inhibitor. The present invention therefore provides a pharmaceutical formulation comprising (i) L-dopa and/or a dopamine agonist and (ii) a pharmaceutically acceptable selective inhibitor of nNO synthase, together with a pharmaceutically acceptable carrier or diluent. Preferred formulations comprise L-dopa either with or without a dopamine agonist. Appropriate pharmaceutically acceptable carriers and diluents are those described above.

The L-dopa may be administered in the form of a pharmaceutically acceptable salt. Appropriate salts are those formed with the acids and bases described above. The L-dopa may alternatively be administered in the form of a pharmaceutically acceptable derivative, e.g. a pharmaceutically acceptable ester.

The dopamine agonist is typically any pharmaceutically acceptable compound which mimics the effects of dopamine in the body and which binds to dopamine receptors. Preferably, the dopamine agonist binds to D2 receptors. Examples of dopamine agonists include bromocryptine, pergolide, ropinirole, pramipexole and cabergoline. These compounds are commercially available for the treatment of Parkinson's disease. Alternative dopamine agonists may also be used. The dopamine agonists may be used in the form of a salt. Appropriate salts are those formed with the acids and bases described above with regard to the NO synthase inhibitors.

Dopamine agonists can be identified by screening, for example using a whole or part animal assay. Thus, for example, a candidate compound may be administered to a selected group of animals, e.g. animals having suppressed dopamine levels, and the resulting effects observed. Control experiments may be carried out using (a) a known dopamine agonist such as ropinirole (positive control: 100% agonist activity) and (b) placebo (negative control: zero agonist activity).

Typically as used herein, a dopamine agonist provides at least 50%, e.g. at least 80% or substantially 100% of the agonist activity observed when ropinirole is used in the above assay.

Where the formulation contains L-dopa, it may also comprise (iii) a peripheral decarboxylase inhibitor. Peripheral decarboxylase inhibitors prevent or reduce the peripheral conversion of L-dopa into dopamine by blocking the peripheral dopa-decarboxylase (DDC) enzyme. This in turns ensures a higher quantity of L-dopa reaches the brain. In the context of this invention, peripheral means extra cerebral.

Examples of peripheral decarboxylase inhibitors include carbidopa and benserazide which are both commercially available for the treatment of Parkinson's in combination with L-dopa. Alternative peripheral decarboxylase inhibitors may also be used.

Inhibitors of peripheral decarboxylase can be identified by screening using a whole or part animal assay. For example, a whole mouse assay may be used which comprises administering a candidate compound, together with L-dopa, to a selected group of animals and observing the resulting effect. Control experiments may be carried out using (a) a known peripheral decarboxylase inhibitor such as carbidopa, together with L-dopa, and (b) using L-dopa alone. Comparison of the results of the candidate compound experiment and controls (a) (positive control: inhibition of DDC) and (b) (negative control: zero inhibition) can then be carried out. For example, the time period over which the L-dopa affects the brain of the animal may be determined for each case.

Typically, as used herein, a peripheral decarboxylase inhibitor is a compound which provides at least 50%, for example at least 80% or substantially 100% of the inhibition observed with carbidopa in the above assay.

The peripheral decarboxylase inhibitor may be administered in the form of a pharmaceutically acceptable salt. Appropriate salts are those formed with the acids and bases described above.

The invention also provides the use of (i) L-dopa and/or a dopamine agonist and (ii) a pharmaceutically acceptable selective inhibitor of nNO synthase, optionally together with (iii) a peripheral decarboxylase inhibitor, in the manufacture of a medicament for use in the treatment of Parkinson's disease.

Also provided is a method of treating Parkinson's disease in a subject, which method comprises the administration to the said subject of (i) an effective amount of L-dopa and/or an effective amount of a dopamine agonist and (ii) an effective amount of a pharmaceutically acceptable selective inhibitor of nNO synthase and optionally (iii) an effective amount of a peripheral decarboxylase inhibitor. Typically, a safe and effective amount of each compound is administered.

Also provided is an agent for the treatment of Parkinson's disease, comprising (i) L-dopa and/or a dopamine agonist, (ii) a pharmaceutically acceptable selective inhibitor of nNO synthase, and optionally (iii) a peripheral decarboxylase inhibitor.

The formulations of the invention are typically provided in a format which provides a daily dose of NO synthase inhibitor as described above. Where administration is in combination with L-dopa, a therapeutically effective amount of L-dopa is administered. A typical daily dose is from about 0.1 to 50 mg per kg of body weight, according to the age, weight and conditions of the subject to be treated, the type and severity of the Parkinson's disease and the frequency and route of administration. Preferably, daily dosage levels are from 1 mg to 50 mg, for example from 2 to 20 mg.

Where administration is in combination with a dopamine agonist, a therapeutically effective amount of dopamine agonist is administered. A typical daily dose is from about 0.01 to 10 mg per kg of body weight, according to the activity of the specific agonist, the age, weight and conditions of the subject to be treated, the type and severity of the Parkinson's disease and the frequency and route of administration. Preferably, daily dosage levels are from 0.02 mg to 5 mg, for example from 0.05 to 1 mg. For example, from 0.02 mg to 1 mg or from 0.05 to 0.5 mg per kg.

Where administration is in combination with a peripheral decarboxylase inhibitor, a therapeutically effective amount of peripheral decarboxylase inhibitor is administered. A typical daily dose is from about 0.1 to 50 mg per kg of body weight, according to the activity of the specific inhibitor, the age, weight and conditions of the subject to be treated, the type and severity of the Parkinson's disease, the amount of L-dopa to be administered and the frequency and route of administration.

Preferably, daily dosage levels are from 1 mg to 50 mg, for example from 2 to 20 mg. For example, from 1 mg to 50 mg, 2 to 20 mg, e.g. 5 to 20 mg or 8 to 20 mg per kg.

Where L-dopa and dopamine agonist are administered in combination, the dosage of one or both compounds may be reduced. In particular, L-dopa may be administered in a reduced amount, eg of from 0.05 to 30 mg eg 0.1 to 20 mg, 0.5 to 10 mg or from 0.5 to 5 mg per kg. In this embodiment any peripheral decarboxylase inhibitor may also be administered in a reduced amount, e.g. of from 0.05 to 30 mg eg 0.1 to 20 mg, 0.5 to 10 mg or from 0.5 to 5 mg per kg.

The selective nNO synthase inhibitor may be administered simultaneously, sequentially or separately from L-dopa. Thus, the present invention provides a product comprising L-dopa and a pharmaceutically acceptable inhibitor of NO synthase for simultaneous, separate or sequential use in the treatment of Parkinson's disease. A dopamine agonist and/or a peripheral decarboxylase inhibitor may also be administered in combination with these compounds, either simultaneously, separately or sequentially.

The selective nNO synthase inhibitor may also be administered simultaneously, sequentially or separately from the dopamine agonist. Thus, the present invention provides a product comprising a dopamine agonist and a pharmaceutically acceptable inhibitor of NO synthase for simultaneous, separate or sequential use in the treatment of Parkinson's disease. L-dopa, and optionally a peripheral decarboxylase inhibitor may also be administered in combination with these compounds either simultaneously, separately or sequentially.

The invention also provides the use of a pharmaceutically acceptable selective inhibitor of NO synthase in the manufacture of a medicament for co-administration with L-dopa and/or a dopamine agonist in the treatment of Parkinson's disease. In addition, the present invention provides the use of L-dopa and/or a dopamine agonist in the manufacture of a medicament for co-administration with a pharmaceutically acceptable selective inhibitor of nNO synthase, in the treatment of Parkinson's disease. Where L-dopa is administered, this may be co-administered with a peripheral decarboxylase inhibitor.

Where the medicaments are to be administered separately, each compound is typically separately formulated into the one of the dosage formats described above. The same or different dosage formats may be used for each compound. The appropriate dosages of each compound are those described above with regard to the pharmaceutical formulations. Optionally, two or more medicaments may be administered simultaneously in a single formulation, whilst at least one medicament is administered separately.

The administration of each of the compounds may be carried out in any order. For example, the L-dopa may be administered substantially simultaneously with a peripheral decarboxylase inhibitor, whilst the selective nNO synthase inhibitor may be administered either substantially simultaneously or afterwards. Similarly, the dopamine agonist may be administered substantially simultaneously with, or before, the selective nNO synthase inhibitor. In the context of this invention, substantially simultaneously means within 2 hours, preferably within 1 hour or within 30 minutes. Thus, the peripheral decarboxylase inhibitor is, for example, administered up to 2 hours, for example up to 1 hour prior to L-dopa.

Typically each medicament is administered within a period of up to 24 hours, preferably up to 12 hours or up to 6 hours. For example, the L-dopa may be administered up to 24 hours, for example up to 12 hours or up to 6 hours, before the NO synthase inhibitor, or the NO synthase inhibitor may be administered up to 24 hours, for example up to 12 hours or up to 6 hours, before L-dopa. Similarly, the peripheral decarboxylase inhibitor may be administered up to 24 hours, for example up to 12 hours or up to 6 hours before, or after, L-dopa.

The following Examples illustrate the invention.

### REFERENCE EXAMPLE

In the following procedure, all NOS inhibitors (nNOSi) were administered subcutaneously. All other drugs with the exception of 7-nitroindazole were dissolved in deionised water or for oral administration dissolved in deionised water containing 10% sucrose and were administered orally. 7-nitroindazole was dissolved in a 30:70 v/v in water and DMSO. In experiments using ropinirole, animals were pre-treated with a single oral dose of domperidone (5 mg·kg⁻¹) as an anti-emetic, 30 min before. Carbidopa and domperidone were given as suspension in deionised water and 10% sucrose.

Induction of Motor Deficits and Priming for Dyskinesia Induction

Adult common marmosets of either sex (Callithrix Jacchus; n=6), weighing between 310 to 380 g were used in this study. Marmosets were housed either in pairs or individually at a temperature of 25±1_ C with 50% relative humidity on a 12-hour light-dark cycle. Animals were fed once daily on a diet of fresh fruit, nuts, Mazuri food pellets, and a supplement of vitamin D3 and had free access to water. On experimental days, the animals were fed following completion of behavioural testing.

MPTP Treatment

Marmosets were treated with 1-methyl-4-phenyl-1,2,3,6-tetrahydropyridine hydrochloride (2.0 mg·kg⁻¹ subcutaneously; MPTP-HCl; Research Biochemicals International; dissolved in 0.9% sterile saline) daily for 5 consecutive days. This treatment regimen induces a persistent and stable syndrome of akinesia, bradykinesia, rigidity, postural abnormality, incoordination and postural tremor (Jenner and Marsden, 1986; Pearce et al., 1995). Animals were hand fed on a cocktail of Mazuri marmoset jelly, dried milk and pureed bananas until body weight had returned towards pre-treatment levels.

Following the last administration of MPTP, all animals showed marked motor deficits, displaying an abnormal hunched posture, akinesia, rigidity, loss of vocalization, and postural tremor. Five weeks following MPTP treatment, the animals had recovered some motor function and were able to independently groom, feed, and drink. Experiments were carried out in accordance with the “Animals (Scientific Procedures) Act 1986” and Home Office regulations (Licence no. PPL 3563).

Priming for Dyskinesia Induction

Eight weeks following MPTP-treatment, all animals (n=6) were treated for 30 to 40 days with 12.5 mg·kg⁻¹ L-DOPA methyl ester (Sigma) plus 12.5 mg·kg⁻¹ carbidopa (Merck Sharp & Dohme) by oral gavage on a daily basis. Carbidopa was administered 30 to 45 min prior to L-DOPA treatment (Pearce et al., 1995). When animals became dyskinetic, daily dosing with a combination of L-DOPA/carbidopa was changed to a once a week treatment. At this point and on, once a week L-DOPA/carbidopa treatment produced similar level of dyskinesia to that produced at the end of the daily priming period.

### Examples 1 to 8

The effects of nitric oxide inhibitors (NOSi), L-nitroarginine (L-NAME; Sigma), Lw-propyl arginine (LPA; Tocris-Cookson) or 7-nitroindazole (7-NI; Sigma) on L-DOPA induced changes in motor activity, motor disability and dyskinesia (chorea and dystonia) was studied. L-Name and LPA are not selective inhibitors of nNOS, while 7-NI is a selective inhibitor of nNOS. L-DOPA primed MPTP treated animals of the Reference Example (n=6) were co-administered with either, 12.5 mg·kg⁻¹ L-DOPA plus 10% sucrose (Example 1) or L-DOPA plus nNOSi (L-NAME: Examples 3 to 5; LPA: Examples 6 and 7; and 7-NI Example 8) 30 to 45 min following 12.5 mg·kg⁻¹ carbidopa administration. The treatments given in each Example are summarized in Table 1 below.

TABLE 1

| Example | L-Dopa (mg kg⁻¹) | nNOSi/amount (mg kg⁻¹) |
|---|---|---|
| 1 | none | none |
| 2 | 12.5 | none |
| 3 | 12.5 | L-NAME/2 |
| 4 | 12.5 | L-NAME/6 |
| 5 | 12.5 | L-NAME/20 |
| 6 | 12.5 | LPA/2 |
| 7 | 12.5 | LPA/6 |
| 8 | 12.5 | 7-NI/20 |

Behavioural Assessment

Motor Activity: Motor activity was measured in test cages equipped with an array of 8 infra red photo sensors (perspex fronted aluminium cages, 50×60×70 cm), The number of beam interruptions due to movement was counted and summed in bins of 10 minute interval using an analogue to digital converter attached to an Intel-based PC. Motor activity was measured over a period of 6 hours and expressed both as time course measuring the number of beam interruption occurring in 10 min time segments, or as the total number of beam interruptions over the test period.

Motor Disability: The following observer rating scale was used to assess motor disability each day: alertness (0 to 2); checking movements (0 to 2); posture (0 to 4); balance (0 to 3); motility (0 to 2); reactions to stimuli (0 to 3) and vocalization (0 to 2). The disability score was obtained by addition of individual scores for each parameter observed. Score zero indicates a normal score, score 18 indicates a maximum motor disability. On experimental days, motor disability was scored every 10 to 20 min for a period of up to 240 min. The motor disability scores were either presented graphically as time-course graphs or expressed as the mean total disability for each experimental day.

Dyskinesia: Abnormal movements following chronic L-DOPA treatment, in the form of chorea (rapid random flicking movements) and dystonia (abnormal sustained posturing) were scored by observer rating. Each parameter was scored on a scale of 0 (absence of dyskinesia) to 4 (marked continuous dyskinetic activity replacing normal behaviour) as previously reported (Pearce et al., 1995). Measurement of dyskinesia consisting of a combination of chorea and dystonia was also scored. This was again rated on a scale of 0 to 4. Dystonia, chorea and dyskinesia scoring was performed simultaneously with motor disability, when animals were being monitored for motor activity.

Results

Results are expressed as mean ±SEM. Data from motor activity was analysed using a repeated measures one way ANOVA for comparison of the effect of different doses of NOSi with vehicle-treated controls at each time point. On obtaining a significant F-value, a Newman-Keuls multiple comparison test was performed. The non-parametric motor disability, chorea and dystonia scores were compared using a Freidman's test followed by Dunn's multiple comparison test.

FIG. 1 depicts the results of the behavioural tests on the vehicle (Example 1), L-dopa alone (Example 2) and Examples 3 to 5 containing 2, 6 or 20 mg/kg L-NAME. The results show a marked decrease in motor activity and also a decrease in choria, dystonia and motor disability for L-NAME treated animals compared with those treated with L-dopa alone. Similar results are presented in FIG. 2 for Examples 6 and 7 using LPA and in FIG. 3 for Example 8 using 7-NI.

FIGS. 4 and 5 provides a summary of the FIGS. 1 and 2 results, showing total motor activity, total motor disability and total dyskenia for each of Examples 1 to 7 (Examples 2 to 7 for total dyskenia).

### Examples 9 and 10

In a further experiment, the effects of NOSi on the motor effects produced by the DA D3/D2 agonist, ropinirole (GSK) 100 μg·kg⁻¹, p.o plus 1 mg·kg⁻¹ domperidone, used as an anti-emetic, was determined. The treatments used were those set out in Table 2 below.

TABLE 2

| Example | Ropinirole (mg kg⁻¹) | L-NAME (mg kg⁻¹) |
|---|---|---|
| 9 | 0.3 | none |
| 10 | 0.3 | 20 |

The treated animals were subjected to the behavioural assessments described above and the results are depicted in FIG. 6. FIG. 6 shows the effectiveness of L-NAME in reducing dopamine agonist induced dyskinesia. Comparison of FIG. 6 with FIG. 1 demonstrates the higher activity of L-NAME on L-DOPA induced dyskinesia, compared to dopamine agonist induced dyskinesia.

### Example 11

The selective eNOS inhibitor N5-(1-iminomethyl)-L-ornithine 2HCl, (“L-NIO”), was tested using the same test protocol as in the Reference Example and Examples 1-8. The results are depicted graphically in FIG. 7, wherein the locomotor activity results are in FIG. 7a, the motor disability results in FIG. 7b, the chorea results in FIG. 7c, and the dystonia results in FIG. 7d. These results show that the selective eNOS inhibitor tested did not reduce L-Dopa induced dyskinesia.

### Example 12

The selective iNOS inhibitors S-(2-aminoethyl)-isothiourea (“S-(2-aminoethyl)-ITU”), and S,S′-1,3-Phenylene-bis(1,2-ethanediyl)-bis-isothiourea (“1,3 PB-ITU”), were tested at 6 mg/kg s.c by the test protocol as in the Reference Example and Examples 1-8. The results are depicted graphically in FIGS. 8 (1,3 PB-ITU) and 9 (S-(2-aminoethyl)-ITU), wherein the locomotor activity results are in FIGS. 8a and 9a, the motor disability results in FIGS. 8b and 9b, the chorea results in FIGS. 8c and 9c, and the dystonia results in FIGS. 8d and 9d. These results show that the selective iNOS inhibitors tested did not reduce L-Dopa induced dyskinesia.

### Example 13

The selective nNOS inhibitor L-N5-(1-imino-3-butenyl)-ornithine (“Vinyl L-NIO”), was tested at 6 mg/kg using the same test protocol as in the reference Example and Examples 1-8. The results are depicted graphically in FIG. 10, wherein the locomotor activity results are in FIG. 10a, the motor disability results in FIG. 10b, the chorea results in FIG. 10c, and the dystonia results in FIG. 10d. These results show that, like 7-NI in Example 8, the selective nNOS inhibitor tested reduced L-Dopa induced dyskinesia.

1. A method of preventing dyskinesia induced by L-dopa or a dopamine agonist comprising administering a pharmaceutically acceptable selective inhibitor of neuronal NO synthase.

2. A medicament comprising (i) a pharmaceutically acceptable selective inhibitor of neuronal NO synthase and (ii) L-dopa or a dopamine agonist, for concurrent, separate or sequential use in the treatment of Parkinson's Disease while reducing dyskinesia induced by said L-dopa or dopamine agonist.

3. A medicament as claimed in claim 2 wherein the medicament is for use in treating dyskinesia established in a subject as a result of repeated administration to the subject of L-dopa or a dopamine agonist.

4. A method of treatment of dyskinesia induced in a subject undergoing therapeutic administration of L-dopa or a dopamine agonist, comprising administering to the subject an effective amount of a pharmaceutically acceptable selective inhibitor of neuronal NO synthase.

5. A method as claimed in claim 4 wherein the dyskinesia is established in the subject as a result of repeated administration to the subject of L-dopa or a dopamine agonist.

6. In a method for treating Parkinson's Disease by administration of L-dopa or a dopamine agonist to a subject suffering Parkinson's Disease, the improvement comprising administering to the subject an amount of a pharmaceutically acceptable selective inhibitor of neuronal NO synthase effective to reduce dyskinesia in said subject induced by said L-dopa or dopamine agonist.

7. The improvement as claimed in claim 6 wherein the dyskinesia is established in the subject as a result of repeated to the subject of L-dopa or a dopamine agonist.

8. A pharmaceutical composition, product, or package in unit dosage form, comprising (i) a pharmaceutically acceptable selective inhibitor of neuronal NO synthase and (ii) L-dopa or a dopamine agonist, and (iii) one or more pharmaceutically acceptable diluents or carriers for (I) and/or (ii), for simultaneous, concurrent, separate or sequential use for the treatment of Parkinson's Disease.

9. A pharmaceutical composition, product or package as claimed in claim 8 additionally comprising a peripheral decarboxylase inhibitor.

10. A pharmaceutical composition, product or package as claimed in claim 9, wherein the peripheral decarboxylase inhibitor is carbidopa or benserazide.
